(19) United States
(12) Patent Application Publication
(10) Pub. No.: US 2022/0160491 A1
(43) Pub. Date: May 26, 2022

(54) BREAST IMPLANT INSERTION DEVICE AND METHOD

(71) Applicants: Neil J. ZEMMEL, Manakin-Sabot, VA (US); Kevin GRUBB, Virginia Beach, VA (US)

(72) Inventors: Neil J. ZEMMEL, Manakin-Sabot, VA (US); Kevin GRUBB, Virginia Beach, VA (US)

(21) Appl. No.: 16/953,799

(22) Filed: Nov. 20, 2020

(51) Int. Cl. A61F 2/00; A61F 2/12

(52) U.S. Cl. CPC A61F 2/0095; A61F 2/12

(57) ABSTRACT

A breast implant insertion device and method includes a shell having an open and a chamber with an implant arranged in the chamber and a flexible sheath removably connected with the shell. A lubricant is inserted into the shell to coat the implant which is then transferred to the sheath. The sheath is separated from the shell. An opening is formed in a distal end of the sheath and the sheath is squeezed to eject the implant from the sheath for insertion into a patient without any contact of the implant.

### BACKGROUND OF THE INVENTION

Breast implant surgery has been performed for many years to augment the female breast for both cosmetic and reconstructive purposes. To date, implant-based breast surgery generally requires the surgeon to manually handle the implant. Implants are constructed of a flexible silicone rubber shell filled with either saline solution or cohesive silicone gel. Implants are manufactured in various shapes and sizes, and the surgeon selects the appropriate device for each patient's needs. During the implant procedure, an incision is made near or on the breast to provide access. A pocket is surgically created beneath the soft tissue of the breast and chest wall for the implant to reside in. The surgeon inserts the implant via the incision to position the implant within the pocket. Breast implant surgery requires meticulous sterile technique as the risk of infection and longer-term complications are significantly increased if implants are handled improperly.

A large body of research has shown that microbial contamination of the breast implant during placement leads to both short and long term complications. During the past decade a large body of evidence within the plastic surgery literature has shown that even a small amount of bacterial contamination during implant placement can lead to complications. Contamination of the implant can occur during any step of the procedure, but most likely occurs during transfer of the implant from the packaging to placement within the soft tissue pocket.

The most significant short-term complication is acute infection which often requires emergency explant surgery in conjunction with a long course of antibiotics. A long-term complication due to bacterial contamination is capsular contracture which is thickening and tightening of the breast implant capsule around the device. It is generally thought that a small amount of bacterial contamination is introduced during implant placement. This minute amount of bacteria is not enough to cause a robust infection, but instead causes a chronic inflammation around the implant leading to contracture. Severe capsular contracture results in distortion of breast shape, thinning of the overlying tissue, and chronic pain. Capsular contracture is often treated with complex revisional surgery or implant removal altogether. Despite the most up to date treatments for capsular contracture, recurrence rates are high. It is therefore paramount that bacterial contamination of the implant be avoided. Surgeons employ a number of maneuvers during the procedure to reduce the risk of contamination. These include changing surgical gloves frequently, using multiple antibiotic irrigations within the pocket, covering the skin around the incision with an antimicrobial barrier, using antimicrobial barriers over the nipple, choosing an inframammary incisional approach, and finally handling the implant as little as possible.

The present invention relates to an improved breast implant insertion device and procedure which eliminates any contact of the implant by the surgeon or patient skin surface. It fully eliminates handling of the implant by the surgeon or accidental handling of the implant by surgical assistants and scrub techs.

### BRIEF DESCRIPTION OF THE PRIOR ART

Various breast implant insertion devices are known in the prior art. For example, EP patent No. 3087951 discloses a silicone implant introduction device includes a tube which receives an implant and a plunger which is operable to force the implant from the tube into a patient. Another type of implant insertion device is disclosed in U.S. Pat. Nos. 9,925,028 and 10,575,936 which disclose a bag having a closed end and an open end with a slit extending from the open end to receive an implant. The slit is then closed or sealed and the bag is compressed to force the implant from the open end into a patient.

While the prior devices operate satisfactorily, they still require that the implant be handled by a surgeon either by manually transferring the implant into a tube or into a bag. Thus, the risk of contamination or overt infection still exists. Therefore, the prior devices are not completely enclosed sterile systems.

The present invention was developed in order to overcome these drawbacks by providing a complete no-touch breast implant insertion device and method in which the implant may be inserted into a patient without being handled by the surgeon or accidentally contaminated by operating room staff.

### SUMMARY OF THE INVENTION

Accordingly, it is a primary object of the invention to provide a breast implant insertion device that includes a hemispherical hard shell which contains a chamber in which the implant is placed. A flexible sheath is connected with the open rim of the hemispherical shell is sealed for implant insertion. The hemispherical shell has an injection port to infuse a solution or lubricant of the surgeon's choice. The distal end of the sheath is sealed to provide a completely enclosed sterile environment for the implant. The hemispherical shell-sheath device is contained within an outer hard plastic container covered with lidding material or closure for shipping of the device. The sheath is multiply folded in an “accordion” like fashion within the outer shipping container.

The outer container and hemispherical shell are preferably formed of an impact resistant material and the sheath is formed from a flexible synthetic plastic material. The sheath includes a portion which is folded in an “accordion” fashion to allow packaging and shipment, and later deployment upon implant insertion. The hemispherical shell-sheath device is made of transparent material to allow visualization of the implant at all times.

The sheath has a tapered configuration and is sealed to provide a terminally sterile environment. Once the surgeon is ready for implant insertion, lubricant or antibiotic solution is infused into the hemispherical shell via the injection port. The hemispherical shell-sheath device containing the implant is then agitated to completely cover the implant with fluid and lubricate the sheath's inner surface. The sheath is then unfolded and the distal end of the sheath is then cut to the desired width for incision length and implant size.

The hemispherical shell-sheath device is then turned upside down to allow complete transfer of the implant into the sheath. The sheath is then detached from the hard-plastic hemispherical shell. The distal end of the sheath is then placed into the breast incision affording access to the implant pocket. A compression force is applied to the sheath to eject the implant from the sheath directly into the patient. As this point, the implant has never been touched or handled by the surgeon and has had no contact with the skin surface. The breast implant is delivered to the breast pocket without ever having touched any potentially contaminating surface.

The invention also relates to a method for inserting an implant into the breast of a patient without any handling of the implant by the surgeon. The implant is a composite device including a hard plastic hemispherical shell sealed to a flexible plastic sheath in a frustrum shape. This device is shipped in an outer hard plastic shell with sealed lidding material. A fluid such as a lubricant or antibiotic solution is inserted into an injection port attached to the hemispherical shell. This fluid will then surround the implant and provide lubrication. The sheath is unfurled and a portion of the distal end of the sheath is removed to define an opening. The hemispherical shell-sheath device is then placed in a shell up and sheath down position to allow the implant to load in the sheath. The sheath is then disconnected and unsealed from the hard plastic hemispherical shell. A compressive force is applied to the sheath to force the implant from the sheath via the opening and into an incision adjacent the patient's breast without contacting the implant.

The hemispherical shell-sheath device containing the implant is shipped in a container such as an outer box which is unsterile. The box preferably is an outer hard plastic container with a sealed lidding material. This outer container then contains the sterile hard plastic hemispherical-sheath device with breast implant inside.

The sheath preferably has a tapered frusto-conical configuration. The distal end portion of the sheath that is removed thus defines the configuration of the opening which is preferably selected in accordance with the size and configuration of the implant and breast implant incision length.

### BRIEF DESCRIPTION OF THE FIGURES

Other objects and advantages of the invention will become apparent from a study of the following specification when viewed in the light of the accompanying drawing, in which:

FIG. 1 is a partial sectional view of a breast implant insertion device according to the invention arranged in a shipping container;

FIG. 2 is a front plan view of the breast implant insertion device after removal from the shipping container and deployment of the sheath;

FIG. 3 is a perspective view of the device of FIG. 2 after unsealing and removal of the hemispherical shell in preparation for operation; and

FIG. 4 is a perspective view of the device of FIG. 3 with the implant partially ejected from the sheath by compressive forces applied to the sheath.

### DETAILED DESCRIPTION

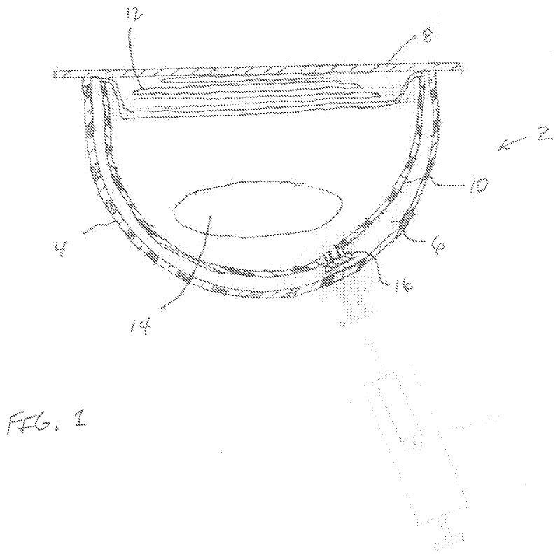

Referring first to FIG. 1, there is shown a package 2 including a shipping container and a breast implant insertion device according to the invention. The package is self-contained and includes an outer container 4 preferably formed of a synthetic plastic. The container is preferably rigid and has a semi-circular cross-sectional configuration to define a chamber 6 within which the sterile breast implant insertion device is arranged. A removable cover 8 is sealed to a rim of the container 4 and closes the chamber to protect the insertion device during shipping while maintaining the insertion device in a sterile condition. It will be appreciated that the container 4 may have other configurations and be formed of different materials. However, the rigidity of the container is advantageous for maximum protection. The cover may also be rigid or it may be in the form of a foil sheet.

The breast implant injection device includes a hemispherical shell 10 and a flexible sheath 12. The shell is preferably formed of a rigid synthetic plastic material whereas the sheath is preferably formed of a flexible synthetic plastic material. As shown in FIG. 1, a breast implant 14 is arranged within the hemispherical hard plastic shell 10 and the sheath 12 is folded within the container 4 beneath the cover 8. The implant is typically formed of a silicone material as is known in the art.

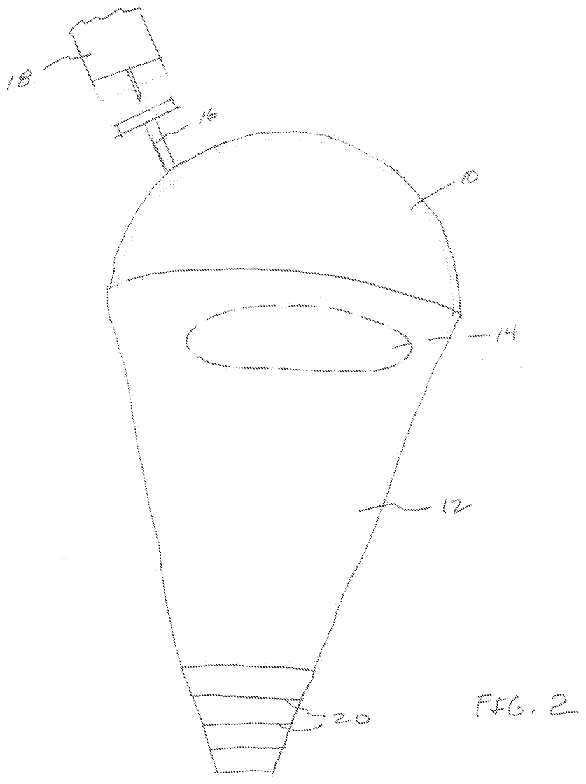

The breast implant insertion device is preferably assembled at a manufacturing facility. An implant 14 of a predetermined size and configuration is arranged in the hemispherical shell 10 and the sheath 12 is attached to the open end of the shell. More particularly, the sheath has a frusto-conical configuration as shown in FIG. 2. The upper or proximal end of the sheath is generally circular and sealed to rim of the hemispherical shell such as by an adhesive. Alternatively, the sheath may be molded together with the shell with the implant arranged in the shell chamber. In either case, the hemispherical shell-sheath device with the implant therein is arranged in the shipping container 4, the sheath 12 is folded as shown in FIG. 1 and the cover is applied. The assembled package is preferably sterilized in a known manner so that the interior of the container, the implant, and the hemispherical shell-sheath insertion device are free of contaminants, bacteria and the like. assembled package is then ready for shipment to a surgical center.

In a preferred embodiment, the breast implant insertion device is assembled without a fluid or lubricant. In this embodiment, an injection port 16 of known type is connected with the hemispherical shell 10 which affords a passage of the lubricant or antibiotic solution to the interior of the sheath. After removal of the sterile implant and injection device from the shipping container and prior to performing an implant procedure, the surgeon uses a syringe 18 to inject the lubricant into the interior of the hemispherical shell-sheath device to surround the implant 14. The device is oscillated or displaced to distribute the lubricant around the entire surface of the implant.

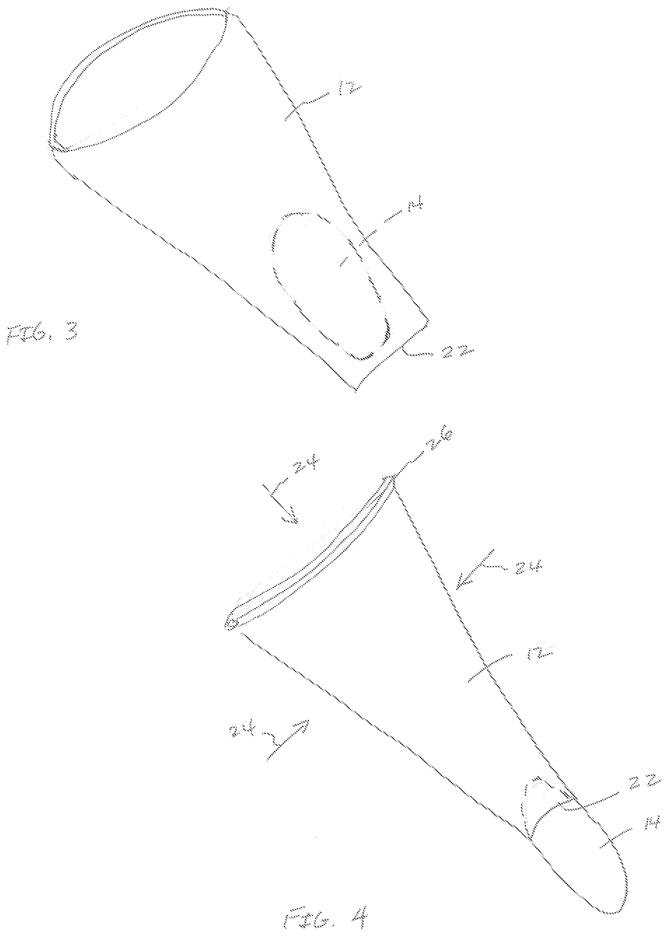

Whether the container is pre-assembled with lubricant or lubricant has been injected into the sheath by the surgeon, the implant insertion device is ready for operation after oscillation. The sheath 12 is separated from the hemispherical shell 10 as shown in FIG. 3. If the sheath proximal end is adhesively connected with the shell, the surgeon merely pulls the sheath and shell away from each other. Other conventional separation techniques may also be provided. For example, for a sheath molded with the shell, a tear strip may be provided between the sheath and the shell. By pulling the tear strip, the surgeon can manually separate the sheath from the shell. Alternatively, a series of indentations may also be provided at the intersection between the sheath and shell. The indentations are broken by pulling the sheath from the shell for separation. Once separated, the surgeon can manually close the open end of the sheath with the lubricant and implant being retained in the sheath as shown in FIG. 4.

At the distal end, the outer surface of the sheath may be provided with a series of spaced parallel lines or indicators 20 that serve as a guide to the surgeon to sever the sheath at a selected line which governs the size of an opening in the distal end of the sheath. This size is selected based upon the configuration or dimensions of the implant as is known in the art.

Once the proper sized opening 22 is provided at the distal end of the sheath as shown in FIG. 3, the implant insertion device is ready for ejecting the implant from the sheath into the patient via an incision made by the surgeon. Referring to FIG. 4, force may be applied to the proximal end and/or to the sides of the sheath as shown by the arrows 24 to force or eject the implant 14 from the sheath and into the patient.

In FIGS. 3 and 4, the sheath is shown with an open proximal end. This may be closed by folding or rolling the proximal end of the sheath as shown at reference numeral 26 in FIG. 4 which also assists in providing the appropriate force to eject the implant from the sheath. If the proximal end is already sealed or closed during the assembly process, it may still be folded to apply a compressive force to the interior of the sheath.

It will be appreciated that after the breast implant injection device is assembled and sterilized, the implant 14 does not come into contact with the surgeon. The “no contact” method thus includes the steps of arranging the implant 14 within the hemispherical shell 10, connecting the sheath 12 with the shell, and folding it in the shipping container 4. The container is sealed with the cover 8 and sterilized. A lubricious fluid is inserted into the hemispherical shell and surrounds the implant. The sheath is unfolded and separated from the container with the implant being displaced into the sheath, and an opening is formed in a distal portion of the sheath, with the size of the opening being determined by the size of the implant. Compressive forces are applied to the sheath to eject the implant from the sheath via the opening for implanting in a patient via an incision provided by the surgeon. The no contact method and implant device minimizes the risk of infection to the patient.

While the preferred forms and embodiments of the invention have been illustrated and described, it will become apparent to those of ordinary skill in the art that various changes and modifications may be made without deviating from the inventive concepts set forth above.

What is claimed is:

1. A breast implant insertion device, comprising
(a) a shell having an open end and a chamber for receiving a breast implant; and
(b) a flexible sheath removably connected with said shell and arranged in said chamber for retaining a breast implant therein.

2. A breast implant insertion device as defined in claim 1, wherein said shell is formed of an impact resistant material.

3. A breast implant insertion device as defined in claim 2, wherein said sheath has a tapered configuration.

4. A breast implant insertion device as defined in claim 4, wherein a narrow end of said sheath contains an opening, whereby a compression force may be applied to said sheath to eject the insert from said sheath directly into a patient without handling the implant.

5. A breast implant insertion device as defined in claim 5, wherein said sheath is formed of a synthetic plastic material.

6. A breast implant insertion device as defined in claim 1, and further comprising an injection port connected with said shell for delivering a fluid from the exterior of said to an interior of said shell and said sheath.

7. A breast implant insertion device as defined in claim 7, wherein said fluid comprises a lubricant.

8. A breast implant insertion device as defined in claim 1, and further comprising a rigid container containing a chamber for receiving said shell, said sheath being folding adjacent to said shell within said container chamber.

9. A breast implant insertion device as defined in claim 8, and further comprising a cover removably connected with said container adjacent to said folded sheath to define a shipping package.

10. A method of inserting an implant into the breast of a patient, comprising the steps of
(a) arranging an implant within a shell having a flexible sheath connected therewith;
(b) inserting a fluid into the shell to surround the implant;
(c) separating the sheath from the shell;
(d) removing a portion of the bottom of the sheath to create an opening; and
(e) forcing the implant from the sheath via the opening to insert the implant into the patient's breast without contacting the implant.

11. A method as defined in claim 10, and further comprising the step of oscillating the sheath to coat the insert with fluid.

12. A method as defined in claim 11, wherein said sheath is tapered in the direction of the opening, and further wherein said removing step includes removing a selected portion of the sheath so that the opening is configured in accordance with the size of the implant.

13. A method as defined in claim 10, wherein the fluid comprises a lubricant.
